(19) United States
(12) Patent Application Publication
(10) Pub. No.: US 2024/0366264 A1
(43) Pub. Date: Nov. 7, 2024

(54) DELIVERY SYSTEM THROUGH A MEDICAL DEVICE

(71) Applicant: Carter Lonsberry, Ellington, CT (US)

(72) Inventor: Carter Lonsberry, Ellington, CT (US)

(21) Appl. No.: 18/631,166

(22) Filed: Apr. 10, 2024

Related U.S. Application Data
(60) Provisional application No. 63/458,247, filed on Apr. 10, 2023.

(51) Int. Cl. A61B 17/34; A61B 17/00

(52) U.S. Cl. CPC A61B 17/3472; A61B 2017/00893

(57) ABSTRACT

A system for delivering medication through a medical device including a passage formed in a body of the medical device, the medical device including an interior defined within the passage and an exterior in fluid communication with the interior, the passage having an inlet and an outlet in fluid communication with the inlet, wherein the passage is configured to fluidly couple a material to a portion of a patient in need of medical treatment.

### CROSS-REFERENCE TO RELATED APPLICATION

This application claims priority to Provisional Application No. 63/458,247 filed Apr. 10, 2023.

### BACKGROUND

The present disclosure is directed to a system and a process for delivering medication through a medical device.

Medical device implants are sterilized and wrapped before surgery. Before the surgery can begin the implants are opened and will sit on a table waiting to be sized and assembled before implanting. The wait time can be a few minutes to several hours before the implants are implanted. This can lead to bacteria forming on the surface of the implant device that has the potential to lead to a surgical site infection. UV lights with varying wavelengths can sterilize the implants. Specifically, a UV lamp placed proximate the implants can be employed to emit UV light onto the implants. UVC light from special germicidal lamps with wavelengths in the range 200 nm to 280 nm kills germs, bacteria, pathogens and viruses and has been used as a means of disinfecting and sanitizing for decades. Another solution to preventing surgical site infection can include coating the implants with a disinfectant spay wipe or gel to help re-sterilize the implant or device.

However once the medical device implant has been implanted, the use of UV lights or disinfectant applications is not available.

What is needed is a system and process for delivering medication via medical implants to promote healing and prevent infection.

### SUMMARY

In accordance with the present disclosure, there is provided a system for delivering medication through a medical device comprising a passage formed in a body of the medical device, the medical device including an interior defined within the passage and an exterior in fluid communication with the interior, the passage having an inlet and an outlet in fluid communication with the inlet, wherein the passage is configured to fluidly couple a material to a portion of a patient in need of medical treatment.

A further embodiment of any of the foregoing embodiments may additionally and/or alternatively include the passage is configured to support a k-wire insertable through the passage from the inlet to the outlet.

A further embodiment of any of the foregoing embodiments may additionally and/or alternatively include the system for delivering medication through a medical device further comprising at least one port formed in the body and in fluid communication with the passage and the exterior.

A further embodiment of any of the foregoing embodiments may additionally and/or alternatively include the ports are placed at a variety of locations along the body.

A further embodiment of any of the foregoing embodiments may additionally and/or alternatively include the ports are configured to deliver the material into bone and/or other body location.

A further embodiment of any of the foregoing embodiments may additionally and/or alternatively include the medical device can be inserted into a bone.

A further embodiment of any of the foregoing embodiments may additionally and/or alternatively include the material is selected from the group consisting of antibiotics, radiation emitting material, chemo treatment, brachytherapy, bone material, bone void filler, and the like.

A further embodiment of any of the foregoing embodiments may additionally and/or alternatively include the medical device includes a screw having a threaded structure along the exterior.

A further embodiment of any of the foregoing embodiments may additionally and/or alternatively include a variety of mechanisms configured to inject the material into the passage can be coupled to the passage.

A further embodiment of any of the foregoing embodiments may additionally and/or alternatively include the material includes a medical treatment material.

In accordance with the present disclosure, there is provided a process for delivering medication through a medical device comprising forming a passage in a body of the medical device; defining an interior within the medical device passage; defining an exterior in fluid communication with the interior, the passage having an inlet and an outlet in fluid communication with the inlet; fluidly coupling the passage to a material; and fluidly coupling the passage to a portion of a patient in need of a medical treatment.

A further embodiment of any of the foregoing embodiments may additionally and/or alternatively include the process further comprising supporting a k-wire insertable through the passage from the inlet to the outlet.

A further embodiment of any of the foregoing embodiments may additionally and/or alternatively include the process further comprising forming at least one port in the body, the at least one port being in fluid communication with the passage and the exterior.

A further embodiment of any of the foregoing embodiments may additionally and/or alternatively include the process further comprising forming ports at a variety of locations along the body.

A further embodiment of any of the foregoing embodiments may additionally and/or alternatively include the process further comprising configuring ports in the body to deliver the material into a bone and/or other body location.

A further embodiment of any of the foregoing embodiments may additionally and/or alternatively include the process further comprising inserting the medical device into a bone.

A further embodiment of any of the foregoing embodiments may additionally and/or alternatively include the material is selected from the group consisting of antibiotics, radiation emitting material, chemo treatment, brachytherapy, bone material, bone void filler, and the like.

A further embodiment of any of the foregoing embodiments may additionally and/or alternatively include the medical device includes a screw having a threaded structure along the exterior.

A further embodiment of any of the foregoing embodiments may additionally and/or alternatively include the process further comprising coupling a variety of mechanisms configured to inject the material into the passage.

A further embodiment of any of the foregoing embodiments may additionally and/or alternatively include the material includes a medical treatment material.

Other details of the system and a process for delivering medication through a medical device are set forth in the following detailed description and the accompanying drawings wherein like reference numerals depict like elements.

### BRIEF DESCRIPTION OF THE DRAWINGS

FIG. 1 is a schematic representation of a medical implant in bone.

FIGS. 2A through 2E are schematic representations of a group of medical implants that are fenestrated.

FIGS. 3A through 3E are a series of schematic representations of a medical implant with a material being injected through the implant.

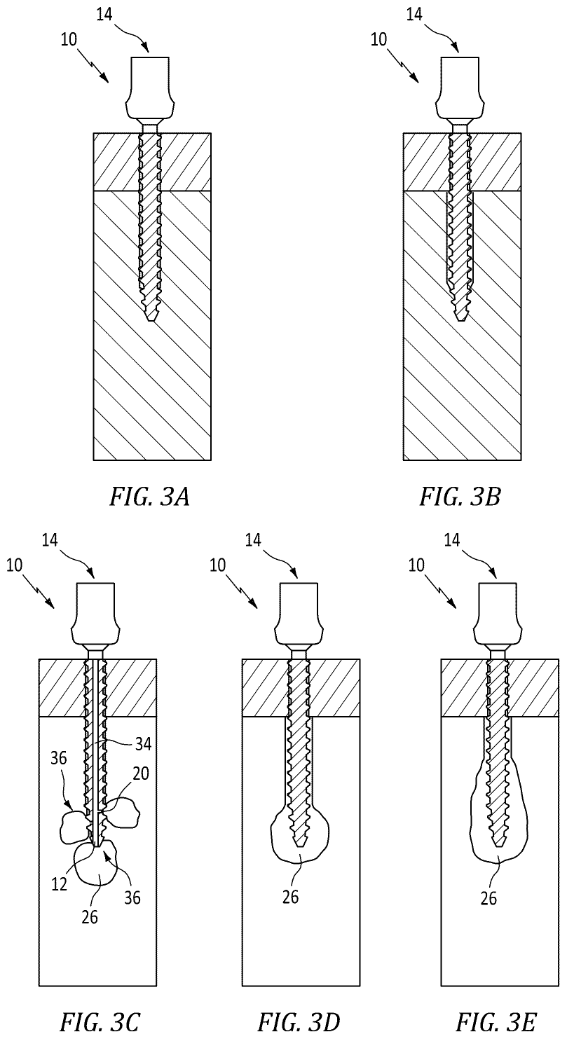

FIG. 4 is a schematic representation of a medical delivery device.

### DETAILED DESCRIPTION

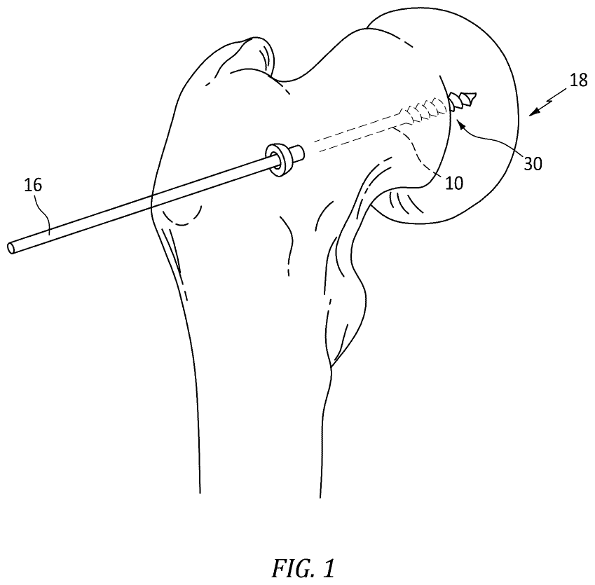

Referring now to the Figures, there is illustrated a variety of exemplary medical implants 10. The medical implants 10 include a passage (cannula) 12 that is formed through a central portion 14. The passage 12 is configured to accept a Kirshner wire (k-wire) 16. The k-wire 16 can be formed in various diameters and lengths. The k-wire 16 can be removed at some point in time during the treatment. The medical implant 10 can be inserted into a bone 18 as seen in FIG. 1.

Fenestrations or simply ports 20 can be formed in the medical implant 10 that are in fluid communication with the passage 12. The ports 20 can be located along the medical implant 10 body 22 and communicate with the exterior 24 of the body 22, such that a material 26 could flow through the passage 12 and discharge out of the ports 20 to the exterior 24.

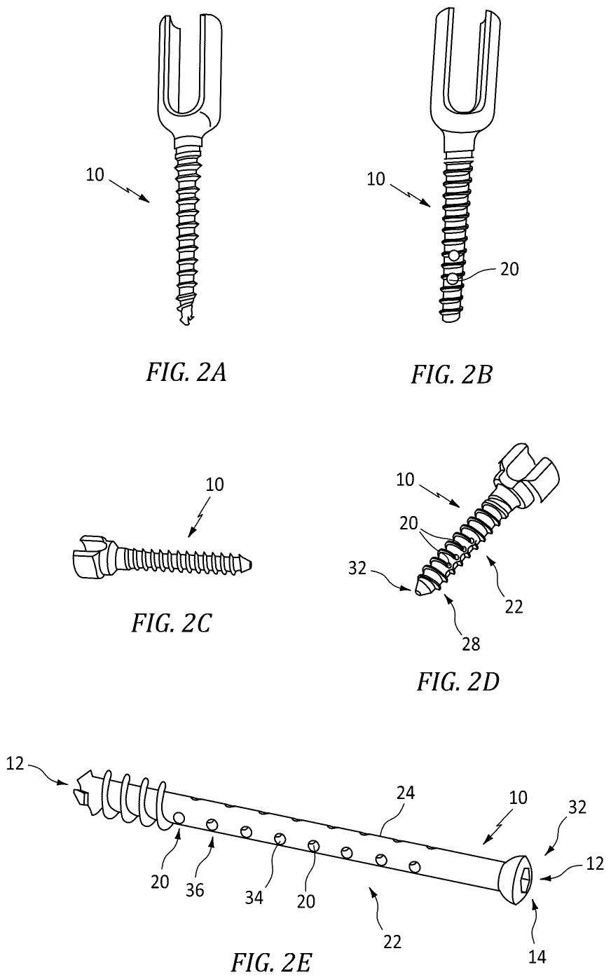

The passage 12 can be utilized not only to allow for the insertion of k-wire 16 but also for the insertion of the material 26 for securing the medical implant 10, see FIGS. 1 and 2E. It is contemplated that the passage 12 can be utilized to insert medical treatment materials 26. The medical treatment material 26 can include but is not limited to the following: antibiotics; radiation emitting material; chemo treatment; brachytherapy; bone material; bone void filler; bone putty; bone matrix material; stem cells; bone substitutes; calcium phosphate; BMP; synthetic bone void filler; P15; RFID chip; poly lactide for time release; plugs; devices used to measure or emit signals; springs; biasing devices to push material through the passage; sensors; accelerometers; neuromonitoring devices; bone density measurement instrument; bone quality measurement instrument; triboelectric nanogenerator for power generation; sensors to measure a range of motion; remote monitoring sensors; steroids; a medical material configured to counter low bone density; anti-inflammatory; pH instruments; chemical instruments; fiducial markers, a mix of medicine with blood, demineralized bone matrix (dbm), synthetic bone and the like.

The ports 20 can be placed at a variety of locations along the body 22 and be configured to deliver the material 26 into bone 18 or other body locations.

In exemplary embodiments, the medical implant 10 can be a screw 28 configured to attach to bone 18 and include a threaded structure 30. The screws 28 can be bone screws 28 that are formed into many shapes and sizes of varying lengths, diameters, head types, cutting flute types and the like. Cannulated screws 28 are screws where a Kirschner wires K-wire 16 (various diameters) is placed first then the screws 28 will slide over the wire 16 for exact screw 28 placement. Once the screw 28 is inserted the wire 16 is removed and thrown away. Partially cannulated screws 28 have the passage 12 that does not extend all the way through the screw body 22. Fenestrated screws are screws with ports 20 in the side of the screw 28 used to increase screw purchase with boney ingrowth or cement.

It is contemplated that a variety of mechanisms can be used to inject the material 26 through the passage 12. Syringes, pumps, IV delivery systems and the like, can be employed to deliver the material 26 into an inlet 32 fluidly coupled to the passage 12 through an interior 34 of the body 22 and flow out of an outlet 36 into the bone 18 or other location that is to be treated.

The material 26 can be shaped to optimize passing through the passage 12. In an exemplary embodiment, the material 26 can be a cylinder-shaped preformed pill. In other embodiments, the material 26 can be shaped in a thumb tack shape or a tee shape. The pills can be pressed into shape within the operating room prior to surgery.

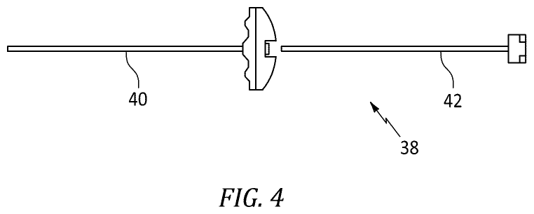

Referring also to FIG. 4, an exemplary medicine delivery device 38. The delivery device 38 can receive the material 26, such as a dose of medication. A delivery tube 40 and a plunger 42 can function to force the material 26 into the passage 12 and ultimately deliver the material 26 to the location of interest.

There has been provided a system and a process for delivering medication through a medical device. While the system and a process for delivering medication through a medical device has been described in the context of specific embodiments thereof, other unforeseen alternatives, modifications, and variations may become apparent to those skilled in the art having read the foregoing description. Accordingly, it is intended to embrace those alternatives, modifications, and variations which fall within the broad scope of the appended claims.

What is claimed is:

1. A system for delivering medication through a medical device comprising:
a passage formed in a body of the medical device, the medical device including an interior defined within the passage and an exterior in fluid communication with the interior, the passage having an inlet and an outlet in fluid communication with the inlet, wherein the passage is configured to fluidly couple a material to a portion of a patient in need of medical treatment.

2. The system for delivering medication through a medical device according to claim 1, wherein the passage is configured to support a k-wire insertable through the passage from the inlet to the outlet.

3. The system for delivering medication through a medical device according to claim 1, further comprising:
at least one port formed in the body and in fluid communication with the passage and the exterior.

4. The system for delivering medication through a medical device according to claim 3, wherein the ports are placed at a variety of locations along the body.

5. The system for delivering medication through a medical device according to claim 1, wherein the ports are configured to deliver the material into bone and/or other body location.

6. The system for delivering medication through a medical device according to claim 1, wherein the medical device can be inserted into a bone.

7. The system for delivering medication through a medical device according to claim 1, wherein the material is selected from the group consisting of antibiotics, radiation emitting material, chemo treatment, brachytherapy, bone material, bone void filler, and the like.

8. The system for delivering medication through a medical device according to claim 1, wherein the medical device includes a screw having a threaded structure along the exterior.

9. The system for delivering medication through a medical device according to claim 8, wherein a variety of mechanisms configured to inject the material into the passage can be coupled to the passage.

10. The system for delivering medication through a medical device according to claim 1, wherein the material includes a medical treatment material.

11. A process for delivering medication through a medical device comprising:
forming a passage in a body of the medical device;
defining an interior within the medical device passage;
defining an exterior in fluid communication with the interior, the passage having an inlet and an outlet in fluid communication with the inlet;
fluidly coupling the passage to a material; and
fluidly coupling the passage to a portion of a patient in need of a medical treatment.

12. The process of claim 11, further comprising:
supporting a k-wire insertable through the passage from the inlet to the outlet.

13. The process of claim 11, further comprising:
forming at least one port in the body, the at least one port being in fluid communication with the passage and the exterior.

14. The process of claim 11, further comprising:
forming ports at a variety of locations along the body.

15. The process of claim 11, further comprising:
configuring ports in the body to deliver the material into a bone and/or other body location.

16. The process of claim 11, further comprising:
inserting the medical device into a bone.

17. The process of claim 11, wherein the material is selected from the group consisting of antibiotics, radiation emitting material, chemo treatment, brachytherapy, bone material, bone void filler, and the like.

18. The process of claim 11, wherein the medical device includes a screw having a threaded structure along the exterior.

19. The process of claim 11, further comprising:
coupling a variety of mechanisms configured to inject the material into the passage.

20. The process of claim 11, wherein the material includes a medical treatment material.
